(19) United States
(12) Patent Application Publication
(10) Pub. No.: US 2009/0208560 A1
(43) Pub. Date: Aug. 20, 2009

(54) Cover material and plaster with cover material

(75) Inventors: Kazunosuke AIDA, Ibaraki (JP); Arata TOSHIMITSU, Ibaraki (JP); Takaaki TERAHARA, Ibaraki (JP); Naruhito HIGO, Saga (JP)

Correspondence Address: THE NATH LAW GROUP, 112 South West Street, Alexandria, VA, 22314, US

(73) Assignee: HISAMITSU PHARMACEUTICAL CO., INC., Saga (JP)

(21) Appl. No.: 12/385,935

(22) Filed: Apr. 24, 2009

Related U.S. Application Data
(63) Continuation of application No. 10/587,832, filed on Oct. 3, 2006.

(30) Foreign Application Priority Data
Jan. 30, 2004 (JP) P2004-024490

(51) Int. Cl. A61K 9/00; A61P 17/00

(52) U.S. Cl. 424/448

(57) ABSTRACT

A cover material adapted to cover the entirety of a patch is provided. The cover material has a support layer and, superimposed on a surface thereof, a pressure sensitive adhesive layer. The patch has a support film with a thickness of 12- to 30 μm and, superimposed thereon, a drug containing layer. The cover material may be attached to the surface of the support film and to the skin around the patch so that the pressure sensitive adhesive layer is brought into contact with edges of the drug containing layer. Additionally, the pressure sensitive adhesive layer contains an adhesive resulting from the polymerization of vinyl acetate or N-vinyl-2-pyrrolidone and a C₈ alkyl (meth)acrylate as essential monomers. Thus, there is provided a cover material that, when a patch medicated with, for example, mesylic acid pergolide is fixed on diseased part, such as, a part of the skin, by covering the patch, is capable of reducing irritation, etc. on the diseased part.

### TECHNICAL FIELD

The present invention relates to a cover material and to a patch with cover material.

### BACKGROUND ART

The occlusive dressing technique (ODT) is a known method for allowing a medicine to reach deep into skin lesions, wherein medicine-applied skin is covered with a thin plastic film and a pressure-sensitive tape is affixed thereto for 2-3 days.

The patch with cover material disclosed in Patent document 1 has the patch completely covered by the cover material when it is attached to the skin, and therefore can be applied for the occlusive dressing technique.
[Patent document 1] Japanese Patent Publication No. 3171935

### DISCLOSURE OF THE INVENTION

### Problems to be Solved by the Invention

However, because the patch with cover material of Patent document 1 has an extremely thin support film (polyester film) forming the patch, problems of deterioration during storage occur, including vaporization and migration of the drug or leakage of the drug despite its being covered with the cover material, and therefore the occlusive dressing technique cannot be effectively implemented. These drawbacks are especially significant when using the anti-Parkinson's drug pergolide mesylate.

Such problems can often be avoided by using support films that are thicker than the support film described in Patent document 1, but the thickness of the support film leads to higher elasticity and greater contact of the support film edges (especially corners) with the skin, thus creating a new problem of increased skin irritation.

It is an object of the present invention to provide a cover material which is adapted to be attached to skin for covering of a patch comprising a drug such as pergolide mesylate, whereby deterioration during storage such as vaporization and migration of the drug or leakage of the drug can be reduced to an acceptable level, while also allowing irritation to the skin to be minimized. It is another object of the invention to provide a patch with cover material having the patch already attached to the cover material.

### Means for Solving the Problems

In order to achieve the aforementioned objects, this invention provides a cover material adapted to be attached to skin in a manner covering over the entirety of a patch, comprising a pressure-sensitive adhesive layer on one side of a support, wherein the patch comprises a drug-containing layer for contacting with the skin provided on a support film with a thickness of 12-30 μm, and wherein the cover material is adapted to be attached to said support film and a region of skin around said patch in such a manner that the pressure-sensitive adhesive layer contacts with the edges of the drug-containing layer, and the pressure-sensitive adhesive layer comprises a pressure-sensitive adhesive obtained by polymerizing vinyl acetate or N-vinyl-2-pyrrolidone and a (meth)acrylic acid alkyl ester with a C8 alkyl group as essential monomer components.

Specifically, the invention provides a cover material comprising a pressure-sensitive adhesive layer on one side of a support, which is adapted to be attached to a region of skin surrounding a patch comprising a drug-containing layer on one side of a support film with a thickness of 12-30 μm and situated so that the drug-containing layer contacts the skin, and which adhesively covers the entirety of the patch with the pressure-sensitive adhesive layer contacting the drug-containing layer exposed at the sides of the patch while being attached to the skin around the patch, wherein the pressure-sensitive adhesive layer comprises a pressure-sensitive adhesive obtained by polymerizing vinyl acetate or N-vinyl-2-pyrrolidone and a (meth)acrylic acid alkyl ester with a C8 alkyl group as essential monomer components.

Since the pressure-sensitive adhesive layer in the cover material of the invention comprises a pressure-sensitive adhesive component composed of a copolymer of specified monomers, when used to cover a patch provided with a drug-containing layer on one side of a support film (hereinafter also referred to simply as “patch”), the pressure-sensitive adhesive layer of the cover material readily wraps around the sides thereof easily producing a condition where the patch is enclosed by the cover material, thereby permitting the occlusive dressing technique to be carried out efficiently.

Also, since the pressure-sensitive adhesive layer in the cover material of the invention comprises a pressure-sensitive adhesive component composed of a copolymer of specified monomers, migration of the drug into the pressure-sensitive adhesive layer is effectively prevented while the pressure-sensitive adhesive layer is in contact with the drug-containing layer, and therefore the released drug becomes concentrated on the skin and percutaneous absorption of the drug is notably improved. Moreover, the problems associated with storage such as drug volatilization and migration or leakage of the drug are minimized. In addition, since using a cover material according to the invention allows the thickness of the support film of the patch to be increased, problems associated with storage such as drug volatilization and migration or leakage of the drug are further reduced.

Furthermore, since the pressure-sensitive adhesive layer contains a pressure-sensitive adhesive component composed of a copolymer of specified monomers, a better balanced is achieved between the elastic modulus of the drug-containing layer of the patch and the elastic modulus of the pressure-sensitive adhesive layer so that the adhesive force on the skin can be adjusted to a suitable level, thus notably reducing skin irritation.

The aforementioned properties are exhibited to an even greater extent when the cover material of the invention is used for skin attachment of a patch comprising pergolide mesylate as the drug. From the same viewpoint, the support is preferably composed of a foamed polymer (polyethylene foam, etc.).

The pressure-sensitive adhesive layer preferably contains a plasticizer (isopropyl myristate, triethyl citrate, liquid paraffin or the like). This allows the adhesive force of the pressure-sensitive adhesive layer to be adjusted for minimal skin eruption or pain upon peeling.

The invention further provides a patch with cover material comprising a cover material provided with a pressure-sensitive adhesive layer on one side of a support and a patch provided with a drug-containing layer on one side of a support film with a thickness of 12-30 μm, attached with the other surface of the support film in contact with the pressure-sensitive adhesive layer so that the pressure-sensitive adhesive layer remains around the periphery of the patch and with the pressure-sensitive adhesive layer contacting the drug-containing layer exposed at the sides of the patch, wherein the pressure-sensitive adhesive layer contains a pressure-sensitive adhesive obtained by polymerizing vinyl acetate or N-vinyl-2-pyrrolidone and a (meth)acrylic acid alkyl ester with a C8 alkyl group as essential monomer components.

Because this manner of patch with cover material employs a cover material according to the invention exhibiting the effect described above, attachment onto skin can be achieved with reduced irritation to the skin, compared to attachment of only a patch onto the skin. Furthermore, since the shelf life of the drug in the patch is extended by the cover material, it is possible to achieve more suitable release of the drug into the skin.

The support film employs a polyethylene terephthalate (hereinafter abbreviated as “PET”) film with a thickness of 12-30 μm. By using such a support film it is possible to maintain greater stability of the drug in the patch. It is also preferred to provide a release liner covering the pressure-sensitive adhesive layer and the drug-containing layer in order to facilitate fabrication, storage and use.

### EFFECT OF THE INVENTION

When the cover material of the invention is used to cover a patch containing a drug such as pergolide mesylate and to affix the patch onto skin, irritation of the patch against the skin can be minimized. This also allows deterioration during storage, including vaporization and migration of the drug or leakage of the drug, to be reduced to an acceptable level. A patch with cover material can also be provided using the cover material.

### BRIEF EXPLANATION OF THE DRAWINGS

FIG. 1 is an enlarged cross-sectional schematic drawing of a patch with cover material according to an embodiment of the invention.

FIG. 2 shows the measurement results for the probe tack test of Example 5.

### EXPLANATION OF SYMBOLS

10 Cover material, 11 Support, 22 Drug-containing layer, 20 Patch, 21 Support film, 100 Patch with cover material.

### BEST MODE FOR CARRYING OUT THE INVENTION

A cover material and patch with cover material according to a preferred embodiment of the invention will now be explained with reference to the accompanying drawings. The dimensional proportions shown in the drawings do no necessarily match the description.

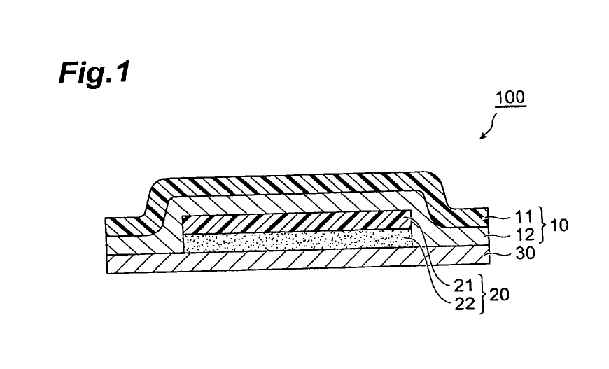

FIG. 1 is an enlarged cross-sectional schematic drawing of a patch with cover material according to an embodiment of the invention.

The patch with cover material 100 according to this embodiment shown in FIG. 1 comprises a cover material 10 composed of a support 11 and a pressure-sensitive adhesive layer 12, a patch 20 composed of a support film 21 and a drug-containing layer 22 with the support film 21 bonded to the pressure-sensitive adhesive layer 12 of the cover material 10, and a release liner 30 attached to and covering the pressure-sensitive adhesive layer 12 and drug-containing layer 22 in a releasable manner.

In the patch with cover material 100, the area of the cover material 10 is wider than the area of the patch 20, and the cover material 10 and patch 20 are bonded in a manner such that the pressure-sensitive adhesive layer 12 of the cover material 10 remains around the periphery of the patch 20. The support film 21 of the patch 20 has a thickness of 12-30 μm. The overall shape of the patch with cover material 100 may be as desired, and so long as the aforementioned structure is provided the shape of the cover material 10 and patch 20 may be, for example, rectangular, circular, elliptical, etc.

The structural material of the support 11 may be a material commonly used for patch supports, and polyethylene foam (hereinafter abbreviated as “PEF”) is particularly preferred.

The pressure-sensitive adhesive layer 12 comprises as the major component a pressure-sensitive adhesive obtained by polymerizing vinyl acetate or N-vinyl-2-pyrrolidone and a (meth)acrylic acid alkyl ester with a C8 alkyl group as essential monomer components. Specifically, the major component used may be: (1) a pressure-sensitive adhesive obtained by polymerizing vinyl acetate and a (meth)acrylic acid alkyl ester with a C8 alkyl group as essential monomer components, with addition of other copolymerizing monomers as necessary, or (2) a pressure-sensitive adhesive obtained by polymerizing N-vinyl-2-pyrrolidone and a (meth)acrylic acid alkyl ester with a C8 alkyl group as essential monomer components, with addition of other copolymerizing monomers as necessary.

As (meth)acrylic acid alkyl esters with C8 alkyl groups there may be mentioned 2-ethylhexyl (meth)acrylate and octyl (meth)acrylate, and as copolymerizing monomers there may be mentioned hydroxyethyl (meth)acrylate, (meth)acrylic acid, (meth)acrylate acid alkyl esters with C1-7 alkyl groups and (meth)acrylic acid alkyl esters with C9-12 alkyl groups. Here, “(meth)acrylic” refers to both methacrylic and acrylic (same hereunder).

The types and contents of the monomers used for the pressure-sensitive adhesive may be selected so as to exhibit a pressure-sensitive adhesive property at the temperature of usage. Specifically, the types of monomers and monomer contents are preferably selected so that the dynamic shear modulus obtained by dynamic displacement at a frequency of 0.01-1 rad/s is 1×10⁵-1×10⁷ dyn/cm² at the temperature of usage (15-40° C., preferably 15-35° C. and more preferably 15-25° C.).

Typical ranges for the monomer ratio of the monomers composing the pressure-sensitive adhesive are shown in Table 1 below.

TABLE 1

| Monomer | Wt % |
|---|---|
| Vinyl acetate or N-vinyl-2-pyrrolidone | 15-35 |
| (Meth)acrylic acid ester with C8 alkyl group | 60-80 |
| Copolymerizing monomer (acrylic acid hydroxy ester, | 0-25 |
| acrylic acid, etc.) | |

The types and contents of the monomers are also preferably determined so that the pressure-sensitive adhesive has a peel strength of 50-300 gF (g force) with a probe tack tester. The pressure-sensitive adhesive layer 12 may be composed of the pressure-sensitive adhesive alone, or other additives may be included in addition to the pressure-sensitive adhesive. In the latter case, the pressure-sensitive adhesive preferably constitutes 70-100 wt % of the pressure-sensitive adhesive layer 12, based on the total weight of the pressure-sensitive adhesive layer 12.

A plasticizer is preferably added to the pressure-sensitive adhesive layer 12 to adjust the pressure-sensitive adhesive force, in consideration of skin irritation and physical properties when the cover material 10 and patch 20 are peeled off from the skin to which they are attached. Particularly preferred as plasticizers are isopropyl myristate, triethyl citrate and liquid paraffin, either alone or in combinations.

The concentration of plasticizer addition may be determined as necessary, but it is preferably added in a range of 5-30 wt % based on the total weight of the pressure-sensitive adhesive layer 12. If the concentration of plasticizer addition is less than 5 wt % it may be difficult to reduce skin irritation, and if it is greater than 30 wt % the pressure-sensitive adhesive force of the pressure-sensitive adhesive layer 12 against the skin will be weakened, tending to result in peeling of the cover material 10 from the skin.

The drug-containing layer 22 has a drug added to a pressure-sensitive adhesive commonly used for patches, and as examples of pressure-sensitive adhesives to be used in the drug-containing layer 22 there may be mentioned acrylic-based pressure-sensitive adhesives, rubber-based pressure-sensitive adhesives, polyurethane-based pressure-sensitive adhesives, silicone-based pressure-sensitive adhesives and gel-forming polymers. As base materials for the pressure-sensitive adhesive there are preferably used natural rubber, synthetic isoprene rubber, polyisobutylene, polyvinyl ether, polyisobutylene, polybutadiene, styrene-butadiene copolymer, styrene-isoprene copolymer, styrene-isoprene-styrene block copolymer, styrene-butylene-styrene block copolymer, agar, gelatin and polyacrylic acid sodium.

A drug is added to the drug-containing layer 22, where the anti-Parkinson's agent pergolide mesylate may be mentioned as a drug to be added, and depending on the purpose of the patch, the drug may be used alone or in a combined formulation in the drug-containing layer 22.

Addition of an absorption accelerator in the drug-containing layer 22 together with the drug can increase permeation of the drug into the skin. Absorption accelerators to be added to the drug-containing layer 22 may be absorption accelerators ordinarily used in poultices and plasters, among which there may be mentioned fatty acid esters (isopropyl myristate, diethyl sebacate, sorbitan monolaurate, glycerin monooleate), sodium oleyl phosphate, sodium lauryl sulfate, octylphenyl ether, lauryl ether, lauroyl diethanolamide, diethanolamide laurate, lauroylsarcosine, oleoylsarcosine sugar ester, lecithin, glycyrrhetin, urea, salicylic acid, methyl salicylate, glycol salicylate, L-menthol, peppermint oil, limonene, calcium thioglycolate, lactic acid, lactic acid esters, olive oil, squalene, lanolin, liquid paraffin, glycerin, aliphatic alcohols (isostearyl alcohol, oleyl alcohol, etc.), acetic acid, Eudragid E and the like, and these may be added depending on the release properties desired for the drug.

As examples of structural materials for the support film 21 there may be mentioned foils, woven fabrics, knitted fabrics and nonwoven fabrics made of synthetic resins including polyesters such as PET, polybutylene terephthalate and polyethylene naphthalate, block copolymer resins composed primarily of ethylene-vinyl acetate copolymer, polyvinyl chloride, PE, polypropylene, polybutadiene, styrene-butadiene or styrene-isoprene, butadiene-styrene-methyl methacrylate copolymer resins, nylon, polyurethane, polyurethane-vinyl chloride copolymer, alkoxyalkyl (meth)acrylate copolymers, polyvinylacetal, polyamide and cellulose derivatives such as rayon, or cotton, hemp, pulp and aluminum films. The support film 21 may have a single-layer structure, or it may be laminated with two or more layers of the aforementioned structural materials.

The support film 21 is most preferably composed of PET. This can further increase the shelf life of the aforementioned drug. The thickness of the support film 21 is 12-30 μm, because a thickness of less than 12 μm may inhibit the effect of the cover material of reducing deterioration during storage including vaporization or migration of the drug or leakage of the drug. On the other hand, if the thickness of the support film 21 is greater than 30 μm, it may be difficult to achieve a suitable condition of sealing with the cover material due to the thickness of the support film itself. The thickness of the support film 21 is preferably 22-28 μm.

The release liner 30 used is preferably, for example, a polyester film made of a polyester such as PET or polyethylene naphthalate, a non-polyester resin film such as nylon, polypropylene, PE or vinyl chloride, or an aluminum foil or paper sheet, having a thickness of about 20-150 μm after release treatment. The release liner 30 may have a single-layer structure, or it may be laminated with two or more layers of the aforementioned structural materials.

The structural materials for the support 11, pressure-sensitive adhesive layer 12, support film 21, drug-containing layer 22 and release liner 30 are as described above, but the structural materials may also be combinations of any of those mentioned above.

A method for fabrication and use of a patch with cover material 100 according to this embodiment will now be explained.

For fabrication of the patch with cover material 100, the pressure-sensitive adhesive used to form the drug-containing layer 22 is prepared first. The drug may be added to the pressure-sensitive adhesive, depending on the purpose of the patch.

After spreading the obtained pressure-sensitive adhesive onto the surface of a sheet for the release liner 30 (here, “sheet” refers to a large-sized sheet prior to cutting to the final shape; same hereunder), it is covered with a sheet for the support film 21 and transferred by contact bonding. A drug-containing layer 22 is thus formed between the release liner 30 sheet and the support film 21 sheet.

Next, the laminate is cut into the desired shape from the support film 21 sheet side to the bonding surface between the release liner 30 sheet and the drug-containing layer 22, and the portions of the support film 21 sheet and drug-containing layer 22 outside of the cut area are peeled off to form multiple patches 20 on the release liner 30 sheet. Each of the multiple patches 20 formed on the release liner 30 sheet is provided with a drug-containing layer 22 on one side of the support film 21, and has the aforementioned shape.

Separately, a pressure-sensitive adhesive obtained by polymerizing vinyl acetate or N-vinyl-2-pyrrolidone and a (meth)acrylic acid alkyl ester with a C10 alkyl group as essential monomer components (preferably a pressure-sensitive adhesive having the properties described above) is dissolved in an organic solvent such as ethyl acetate, hexane or toluene, to prepare a solution for formation of the pressure-sensitive adhesive layer 12 of the cover material 10.

The solution is coated onto a different release liner sheet than the release liner 30 sheet and the organic solvent is removed to form a pressure-sensitive adhesive layer 12 on the release liner sheet. After then attaching a sheet for the support 11 onto the pressure-sensitive adhesive layer 12, the release liner sheet is removed to obtain a cover material 10 sheet having the pressure-sensitive adhesive layer 12 laminated on the support 11 sheet.

The cover material 10 sheet is then bonded with the release liner 30 sheet having multiple patches 20 formed thereon. This results in the pressure-sensitive adhesive layer 12 of the cover material 10 sheet being bonded to the multiple formed patches 20. The bonded laminate that is obtained is then cut to the desired shape to obtain a patch with cover material 100. In this case, cutting out to a larger area than the patch 20 so that the patch 20 is near the center will yield a cover-bearing patch 100 having a form wherein the entire surface of the patch 20 formed on the release liner 30 is covered by the cover material 10.

A solution of the pressure-sensitive adhesive obtained by polymerizing vinyl acetate or N-vinyl-2-pyrrolidone and a (meth)acrylic acid alkyl ester with a C10 alkyl group as essential monomer components may be coated and dried on the release liner 30 sheet having multiple patches 20 formed thereon, prior to attachment of the support 11 sheet thereover and cutting out of the entire laminate.

For use, the release liner 30 of the patch with cover material 100 obtained in this manner is peeled off and the exposed pressure-sensitive adhesive layer 12 and drug-containing layer 22 are attached to the target site of the skin.

Since the patch with cover material 100 of this embodiment has a layer containing a pressure-sensitive adhesive obtained by polymerizing vinyl acetate or N-vinyl-2-pyrrolidone and a (meth)acrylic acid alkyl ester with a C10 alkyl group as essential monomer components, the drug-containing layer 22 containing a drug such as pergolide mesylate can be satisfactorily sealed with the cover material 10. This can stably maintain the drug in the drug-containing layer 22, and permit efficient percutaneous absorption of the drug through the skin during use.

Also, since the pressure-sensitive adhesive layer 12 of the cover material 10 adheres to the support film 21 and drug-containing layer 22 on the patch 20 side, it is possible to reduce irritation against the skin by the edges of the support film 21.

Furthermore, since the elastic modulus of the portions of the cover material 10 adhering to the skin and the portions of the patch 20 adhering to the skin can be adjusted to comparable degrees, the cover material 10 and patch 20 adhere to the skin in a satisfactory balance, thereby allowing irritation against the skin to be reduced.

The above detailed explanation of a preferred embodiment of the invention is naturally not intended to restrict the scope of the invention to this particular embodiment. For example, the cover material 10 of this embodiment may be adhered to a different release liner than the patch 20 for storage.

Also, in this embodiment the drug-containing layer 22 is formed between the release liner 30 sheet and support film 21 sheet by spreading the pressure-sensitive adhesive onto the surface of the release liner 30 sheet and then covering it with the support film 21 and transferring it by contact bonding, but alternatively the pressure-sensitive adhesive may first be spread on the surface of the support film 21 sheet and then covered and contact bonded with the release liner 30 sheet.

Also according to this embodiment, the cover material 10 sheet having the pressure-sensitive adhesive layer 12 laminated on the support 11 sheet is obtained by coating the aforementioned solution onto a different release liner sheet than the release liner 30 sheet, removing the organic solvent to form the pressure-sensitive adhesive layer 12 on the release liner sheet, attaching the support 11 sheet onto this pressure-sensitive adhesive layer 12 and then removing the release liner sheet, but alternatively the solution may be coated onto the support 11 sheet and the organic solvent removed to form the pressure-sensitive adhesive layer 12 on the support 11 sheet.

### EXAMPLES

The present invention will now be explained in greater detail using examples of the invention with the implicit understanding that the invention is not limited to these examples, and that various modifications may be implemented within a range that does not fall outside the technical scope of the invention. Throughout the examples, the “%” values are all weight percentages.

### Example 1

In a solvent there were polymerized 75% 2-ethylhexyl acrylate, 20% vinyl acetate and 5% hydroxyethyl acrylate to obtain a copolymer (hereinafter referred to as “copolymer A”). The solution of copolymer A was coated onto a release liner, and then the solvent was removed by drying to form a pressure-sensitive adhesive layer which was pasted onto a PEF sheet (1.0 mm thickness) as the support to obtain a cover material for Example 1. The pressure-sensitive adhesive layer of this obtained cover material had a thickness of 50 μm, and no physical property problems such as stringing or flapping were observed. The peel strength of the cover material with a probe tack tester was 106.2 gF.

Separately, there was also fabricated a patch comprising a drug-containing layer containing the anti-Parkinson's agent pergolide mesylate sandwiched between a support film (PET film) and a release liner, in the following manner. Specifically, a pressure-sensitive adhesive (Duro-TAK87-4098, product of National Starch & Chemical Co., Ltd.), comprising pergolide mesylate, diethanolamide laurate and lactic acid that had been taken in a mortar and thoroughly mixed and then dissolved in ethyl acetate, was combined with the other components listed in Table 2 to prepare a pressure-sensitive adhesive solution. There were also added ethyl acetate and n-heptane as additional solvents for dissolution of the alicyclic saturated hydrocarbon resin and styrene-isoprene-styrene block copolymer. The mixture obtained in this manner was coated onto a release liner and the solvent was removed by drying, after which it was pasted onto a support film (25 μm-thick PET film) to obtain a patch. The components of the mixture are listed in Table 2.

TABLE 2

| | Content |
|---|---|
| Components of mixture for Example 1 | (wt %) |
| Styrene-isoprene-styrene block copolymer | 23.0 |
| Acrylic pressure-sensitive adhesive (trade name: Duro- | 2.0 |
| TAK 87-4098) | |
| Alicyclic saturated hydrocarbon resin (trade name: | 40.0 |
| ARKON P100) | |
| Liquid paraffin | 15.0 |
| Lactic acid | 6.0 |
| Diethanolamide laurate | 5.0 |
| Pergolide mesylate | 9.0 |

Next, the laminate was cut from the support film side to the release liner surface contacting with the drug-containing layer, and the excess support film and drug-containing layer were discarded. The cover material with the release liner removed was pasted onto the support film side of the patch and the laminate was cut to an appropriate size to obtain a patch with cover material for Example 1. The obtained patch with cover material exhibited adhesion superior to adhesion of the patch alone, and no effect of the presence of the cover material on release of the drug from the patch was observed.

### Example 2

A cover material for Example 2 was obtained in the same manner as Example 1, except that 80% of copolymer A and 20% of isopropyl myristate as a plasticizer were combined and coated onto the release liner. The obtained cover material exhibited no physical property problems such as stringing or flapping of the pressure-sensitive adhesive layer. The peel strength of the cover material with a probe tack tester was 66.4 gF.

A patch with cover material for Example 2 was then produced by the same method as in Example 1. This patch with cover material had satisfactory adhesion and low skin irritation upon peeling.

### Example 3

In a solvent there were polymerized 75% 2-ethylhexyl acrylate, 20% vinyl acetate and 5% acrylic acid to obtain a copolymer (hereinafter referred to as “copolymer B”). A cover material for Example 3 was obtained in the same manner as Example 1, except that 70% of copolymer B and 30% of triethyl citrate as a plasticizer were combined and coated onto the release liner. The pressure-sensitive adhesive layer of this obtained cover material had a thickness of 50 μm, and no physical property problems such as stringing or flapping were observed. The peel strength of the cover material with a probe tack tester was 67.0 gF.

A patch with cover material for Example 3 was then produced by the same method as in Example 1. This patch with cover material had satisfactory adhesion and low skin irritation upon peeling.

### Example 4

In a solvent there were polymerized 80% 2-ethylhexyl acrylate and 20% N-vinyl-2-pyrrolidone to obtain a copolymer. A cover material for Example 4 was obtained in the same manner as Example 1, except that 85% of the obtained copolymer and 15% of isopropyl myristate as a plasticizer were combined and coated onto the release liner. The pressure-sensitive adhesive layer of this obtained cover material had a thickness of 50 μm, and no physical property problems such as stringing or flapping were observed. The peel strength of the cover material with a probe tack tester was 104.8 gF.

A patch with cover material for Example 4 was then produced by the same method as in Example 1. This patch with cover material had satisfactory adhesion and low skin irritation upon peeling.

### Comparative Example 1

A patch with cover material for Comparative Example 1 was fabricated in the same manner as Example 1, except that a woven fabric laminate support was used as the support film for the patch. The woven fabric laminate was obtained by laminating a PET woven fabric with a basis weight of 88.3 g/m³ and a thickness of 0.45 mm onto a PET film with a thickness of 1.8 μm.

### Example 5

A prescribed amount of isopropyl myristate (IPM) was added to copolymer A and the mixture was coated onto a PEF sheet to obtain a cover material. The PEF sheet used here was of two types having thicknesses of 1 mm and 1.25 mm. For comparison, coating was also performed on a PET film with a thickness of 25 μm to examine the effect of the plasticizer.

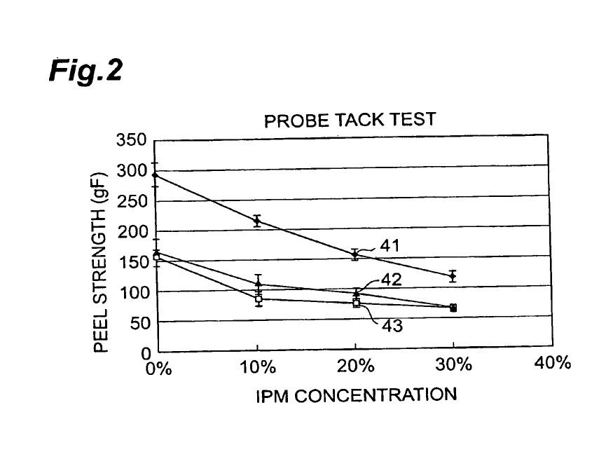

Cover materials with different plasticizer contents were measured for peel strength by probe tack test, and this was used as the index for evaluation of the pressure-sensitive adhesive strength. The results are shown in FIG. 2. The circles 41 represent the 25 μm-thick PET film, the triangles 42 represent the 1.25 mm-thick PEF sheet and the squares 43 represent the 1 mm-thick PEF sheet.

The test results showed that the peel strength was weaker with a large amount of IPM addition, and that the tendency was more notable with the PET film system. Although addition of a plasticizer such as IPM can modify (generally lower) the elastic modulus of the pressure-sensitive adhesive layer of the cover material, alteration of the pressure-sensitive adhesive force (probe tack) by different amounts of plasticizer addition is minimal when using a PEF sheet as the support as in this example. In other words, by using a PEF sheet as the support and adding a plasticizer to the pressure-sensitive adhesive layer, it is possible to modify the elastic modulus while maintaining a roughly consistent pressure-sensitive adhesive force, thereby allowing a satisfactory balance to be achieved with the elastic modulus of the drug-containing layer of the patch, for example.

### Example 6

The patches with cover material of Example 1 and Comparative Example 1 were stored for prescribed periods at prescribed temperatures.

The release liners were then peeled from the patches with cover material and the drug-containing layers were set in the rotating cylinder of an elution tester, facing outward. Next, a round-bottom flask containing 900 mL of purified water was set in the elution tester and the temperature was adjusted to 32° C. The rotating cylinder was immersed in the purified water of the round-bottom flask and rotated at a speed of 50 rpm. A 10 mL portion of eluate was sampled at each time point, the drug concentration in the sample was measured by high-performance liquid chromatography, and the water release at each time point was calculated. The cumulative release rate of pergolide mesylate per 6-hour period was measured in this manner.

The stability of release was evaluated using the value before storage of each sample as the initial value (100%). The results are shown in Table 3.

TABLE 3

| Storage period | Example 1 | Comp. Example 1 |
|---|---|---|
| 25° C., 12 months | 95.6 | 84.5 |
| 30° C., 6 months | 93.9 | 84.2 |
| 40° C., 6 months | 83.7 | 39.1 |
| 60° C., 1 month | 65.9 | 12.7 |

The test results confirmed that the patch with cover material of Comparative Example 1 had a greater reduction from the initial value and an inferior shelf life, compared to the patch with cover material of Example 1.

### Example 7

An organic solvent (ethyl acetate, n-heptane) solution was prepared for a mixture having the same composition as in Table 2 but containing no pergolide mesylate (placebo), and was coated onto a PET film with a thickness of 25 μm, after which the organic solvent was removed to form a pressure-sensitive adhesive layer on the PET film. It was then cut out to an area of 16 cm² or 25 cm². Hereunder, the piece with an area of 16 cm² will be referred to as “placebo patch 1”, and the piece with an area of 25 cm² will be referred to as “placebo patch 2”.

Separately, cover materials 1 and 2 shown in Table 4 below were prepared as the cover materials. Patches with cover material 1 and 2 were then fabricated with the combinations shown in Table 5.

TABLE 4

| Cover | |
|---|---|
| material | Construction |
| Cover | Pressure-sensitive adhesive layer comprising copolymer |
| material 1 | A of Example 1 formed on a 1.25 mm-thick PEF |
| | support (area: 36 cm²). |
| Cover | Pressure-sensitive adhesive layer comprising isopropyl |
| material 2 | myristate as a plasticizer added to copolymer A of |
| | Example 1 (content of plasticizer in pressure-sensitive |
| | adhesive layer: 20 wt %), formed on a 1.25 mm-thick |
| | PEF support (area: 36 cm²). |

TABLE 5

Construction of patch with cover material

| | Cover material | Patch |
|---|---|---|
| Patch with cover | Cover material 1 | Placebo patch 1 |
| material 1 | | |
| Patch with cover | Cover material 2 | Placebo patch 2 |
| material 2 | | |

The patches with cover material 1 and 2 were used for a primary irritation test on human skin. The irritation indices were based on the scale shown in Table 6. For comparison, the same evaluation was conducted for the placebo patch 2 having no cover material. The results are shown in Table 7.

TABLE 6

| Score table | | |
|---|---|---|
| (SI values in | | |
| parentheses) | | |
| − | (0) | No change (identical to surrounding area) |
| ± | (50) | Slight erythema |
| + | (100) | Evident erythema |
| ++ | (200) | Erythema and edema |
| +++ | (300) | Erythema + edema + whealing, mild blistering |
| ++++ | (400) | Blistering |

TABLE 7

| | Irritation index | Irritation index |
|---|---|---|
| Patch with cover | immediately after | 24 hrs after |
| material | peeling (1 hr) | peeling |
| Patch with cover | 55.3 | 15.8 |
| material 1 | | |
| Patch with cover | 37.5 | 15.0 |
| material 2 | | |
| Placebo patch 2 | 38.9 | 5.6 |

The test results revealed irritation indices of 55.3 and 15.8 immediately and 24 hours after peeling for the patch with cover material 1, or in other words, transient irritation immediately after peeling but a sufficiently low value for the irritation index (≦40) after 24 hours, which may be considered suitable for ordinary usage as a patch. With the patch with cover material 2, the irritation indices immediately after and 24 hours after peeling were both below 40, demonstrating that it is possible to provide a formulation having the same high degree of safety on skin as a placebo patch lacking a cover material.

### INDUSTRIAL APPLICABILITY

Using the cover material of the invention to cover a patch containing a drug such as pergolide mesylate and affix the patch onto skin can reduce irritation of the patch against the skin. In addition, deterioration during storage, including vaporization and migration of the drug or leakage of the drug, can be reduced to an acceptable level. A patch with cover material can also be provided using the cover material.

1-10. (canceled)

11. A cover material adapted to cover a patch having a support film and a drug-containing layer, the cover material comprising:
a support layer; and
a pressure-sensitive adhesive layer comprising a pressure-sensitive adhesive provided on a side of the support layer and adapted to be attached to the support film and a region of the skin proximate the patch in such a manner that the pressure-sensitive adhesive layer contacts the edges of the drug-containing layer,
the pressure sensitive adhesive being obtained by polymerizing a monomer selected from the group consisting of vinyl acetate and N-vinyl-2-pyrrolidone and a (meth)acrylic acid alkyl ester with a C8 alkyl group in the presence of at least one co-polymerizing monomer selected from the group consisting of hydroxyethyl (meth)acrylate, (meth)acrylic acid, (meth)acrylic acid alkyl esters with C1-7 alkyl groups, and (meth)acrylic acid alkyl esters with C9-12 alkyl groups,
wherein the pressure-sensitive adhesive layer and the drug-containing layer are each configured such that a drug contained in the drug-containing layer does not migrate into said pressure-sensitive adhesive layer.

12. The cover material according to claim 11, wherein the co-polymerizing monomer is present in an amount of up to about 25% by weight.

13. The cover material according to claim 11, wherein the support film has thickness in the range of from 12 μm to 30 μm.

14. The cover material according to claim 11, wherein the drug containing layer comprises an acrylic pressure-sensitive adhesive and a styrene-isoprene-styrene block copolymer.

15. A patch system, comprising:
a patch comprising
a support film comprising a first and a second surface and being from 12 μm to 30 μm thick, and
a drug-containing layer provided on the first surface of the support film, the drug containing layer comprising an acrylic pressure-sensitive adhesive and a styrene-isoprene-styrene block copolymer; and
a cover material adapted to cover the entire patch, the cover material comprising
a support layer, and
a pressure-sensitive adhesive layer comprising a pressure-sensitive adhesive provided on the second surface of the support film and adapted to cover a region of the skin proximate the patch in such a manner that the pressure-sensitive adhesive layer contacts the edges of the drug-containing layer,
the pressure sensitive adhesive being obtained by polymerizing a monomer selected from the group consisting of vinyl acetate and N-vinyl-2-pyrrolidone and a (meth)acrylic acid alkyl ester with a C8 alkyl group in the presence of at least one co-polymerizing monomer selected from the group consisting of hydroxyethyl (meth)acrylate, (meth)acrylic acid, (meth)acrylic acid alkyl esters with C1-7 alkyl groups, and (meth)acrylic acid alkyl esters with C9-12 alkyl groups,
wherein the pressure-sensitive adhesive layer and the drug-containing layer are each configured such that a drug contained in the drug-containing layer does not migrate into said pressure-sensitive adhesive layer.

16. The patch system according to claim 13, wherein the co-polymerizing monomer is present in an amount of up to about 25% by weight.
